(19) United States
(12) Patent Application Publication
(10) Pub. No.: US 2017/0366773 A1
(43) Pub. Date: Dec. 21, 2017

(54) PROJECTION IN ENDOSCOPIC MEDICAL IMAGING

(71) Applicant: Siemens Aktiengesellschaft, Munich (DE)

(72) Inventors: Atilla Kiraly, Plainsboro, NJ (US); Ali Kamen, Skillman, NJ (US); Thomas Pheiffer, Langhorne, PA (US); Anton Schick, Velden (DE)

(21) Appl. No.: 15/187,840

(22) Filed: Jun. 21, 2016

(51) Int. Cl. H04N 5/372; A61B 1/06; A61B 1/05; A61B 1/04; A61B 1/045; H04N 5/225; A61B 1/00

(52) U.S. Cl. CPC H04N 5/372; H04N 5/2256; A61B 1/0676; A61B 1/00045; A61B 1/043; A61B 1/045; A61B 1/00006; A61B 1/051; G06T 2207/10028; G06T 2207/10068; G06T 2207/10152; G06T 2207/30004

(57) ABSTRACT

A projector in an endoscope is used to project visible light onto tissue. The projected intensity, color, and/or wavelength vary by spatial location in the field of view to provide an overlay. Rather than relying on a rendered overlay alpha-blended on a captured image, the illumination with spatial variation physically highlights one or more regions of interest or physically overlays on the tissue.

### BACKGROUND

The present embodiments relate to medical imaging. In particular, endoscopic imaging is provided.

Endoscopes allow the operator to view tissue using a small device inserted into a patient. Accuracy is important to both guide the endoscope and any tools for performing any surgical operation at the correct location. The use of preoperative computed tomography (CT) volumes, magnetic resonance (MR) volumes, functional imaging volumes, or ultrasound volumes may assist during surgery. In order to assist guidance during surgery, the physical location of the endoscope is registered to a location in the preoperative volume. Previous approaches either involved magnetic or radio frequency tracking the endoscope or by analysis of the video images captured by the endoscopic device. In the latter case, the video feed is analyzed either in real-time or at particular frames in comparison to a virtual rendered view from the preoperative volume.

To improve the registration between the preoperative data and the endoscope, a phase-structured light pattern may be projected from the endoscope in order to compute a depth map. As the endoscope captures frames of video, alternating frames are used to compute a depth map and not shown to the user. Frames shown to the user contain standard illumination, while the phase-structured light pattern is projected during depth-computation frames not shown to the user. This depth map can be used to improve registration performance.

Registration may allow rendering of an overlay from the preoperative data onto the endoscopic video output. Any overlays are performed by post processing and blending a computer rendering with the endoscope image. The overlay may include a target location, distance to target, optimal path, or other information. However, the overlay may block portions of the video from the endoscope. Since the overlay is created as a computer rendering or graphics, the overlay is not physically visible to any other devices when looking at the imaged tissue. The overlay lacks real context, blocks the endoscope video, provides less realistic interactions with the image, and may make overlay errors less obvious to the user. In addition, properly positioning overlays requires knowing the precise optical properties of the endoscopic imaging system to match the view. This correct positioning requires calibrating the endoscope with an image to determine the imaging properties. For example, a “fish-eye” lens distortion is commonly found in endoscopes. Such a distortion is then be applied to the overlay to more precisely account for the view.

### BRIEF SUMMARY

By way of introduction, the preferred embodiments described below include methods, systems, endoscopes, instructions, and computer readable media for projection in medical imaging. A projector in an endoscope is used to project visible light onto tissue. The projected intensity, color, and/or wavelength vary by spatial location in the field of view to provide an overlay. Rather than relying on a rendered overlay, the illumination with spatial variation physically highlights one or more regions of interest or physically overlays on the tissue. Such a solution may eliminate the need to physically model the imaging system of the viewing component or lens as is necessary with a traditional overlay.

In a first aspect, an endoscope system includes a projector on an endoscope. A controller is configured to control a spatial distribution of illumination from the projector onto tissue in a first pattern. A camera on the endoscope is configured to capture an image of patient tissue as illuminated by the spatial distribution of the first pattern. A display is configured to display the image from the camera.

In a second aspect, a method is provided for projection in medical imaging. A target in a field of view of an endoscope is identified. The endoscope illuminates the target differently than surrounding tissue in the field of view and generates an image of the field of view while illuminated by the illuminating.

In a third aspect, a method is provided for projection in medical imaging. A first pattern of structured light is projected from an endoscopic device. The endoscopic device generates a depth map using captured data representing the first pattern of structured light. A second pattern of light is projected from the endoscopic device. The second pattern varies in color, intensity, or color and intensity as a function of location. An image of tissue as illuminated by the second pattern is captured and displayed. The projecting of the first pattern and generating alternate with the projecting of the second pattern, capturing, and displaying.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a diagram of one embodiment of an endoscope system for projection in medical imaging;

FIG. 2 illustrates projecting varying color or intensity light in a field of view;

FIG. 3A shows illumination according to the prior art, and FIGS. 3B-D show spatially varying illumination for imaging; and

FIG. 4 illustrates use of spatially varying illumination for drug activation and/or viewing separate from the endoscopic video;

FIG. 5 is a flow chart diagram of one embodiment for projection in medical imaging; and

FIG. 6 is a flow chart diagram of another embodiment for projection in medical imaging.

### DETAILED DESCRIPTION OF THE DRAWINGS AND PRESENTLY PREFERRED EMBODIMENTS

Standard endoscopes provide views inside the human body for minimally invasive surgery or biopsies. Navigation and guidance may be assisted by image overlays on the displayed screen. However, such a process gives an artificial view from blending of two images. In addition, standard endoscopes use uniform illumination that does not finely adjust to the environment.

Rather than or in addition to relying on virtual or rendered overlays in a displayed image, overlay information is physically performed via projection. The overlays and/or projected information are physically on the target tissue. Using image projection from the actual endoscope itself, regions may be physically highlighted. The light interactions with the tissue and overlays are actually present on the tissue and may be presented in a less obstructing way than with artificial overlays. The physical or actual highlighting of the tissue itself results in the highlighting being not only visible on the display but also visible to other viewers or cameras in the surgical area. By performing a physical projection, the overlay is now visible to other endoscopes, devices, and/or viewers capable of viewing the projected data. Since the overlay is physically present, distortions due to imaging systems such as the endoscope, itself do not need to be considered in displaying the overlay.

The projection gives a fine control of illumination not possible with standard endoscopes and opens a wide variety of applications. Applications involving optimal overlays visible to every camera and/or person in the operating room may benefit. The illumination control by the projection allows the endoscope to be a targeted drug delivery device and offer images with finely controlled illumination. Since the projector tends to have simpler optical properties than the lens system, adapting the projection to be placed on the correct regions is far simpler.

In one embodiment using the projector for registration, a synchronous projection and depth sensing camera is provided. The endoscopic device produces optical and depth mapped images in alternating fashion. A projector produces patterns of illumination in captured frames. In addition to projecting a specific pattern to compute the depth-sensing frame, a projection is performed during the capture of the standard optical image. The projection for optical viewing may highlight a region of interest determined by image processing and registration, such as determining the region of interest from registration with a preoperative CT, MR, X-ray, ultrasound, or endoscope imaging. The projection for optical capture may be used to assist in setting a contrast level for capture by the camera, such as by projecting different intensity light to different locations (e.g., different depths).

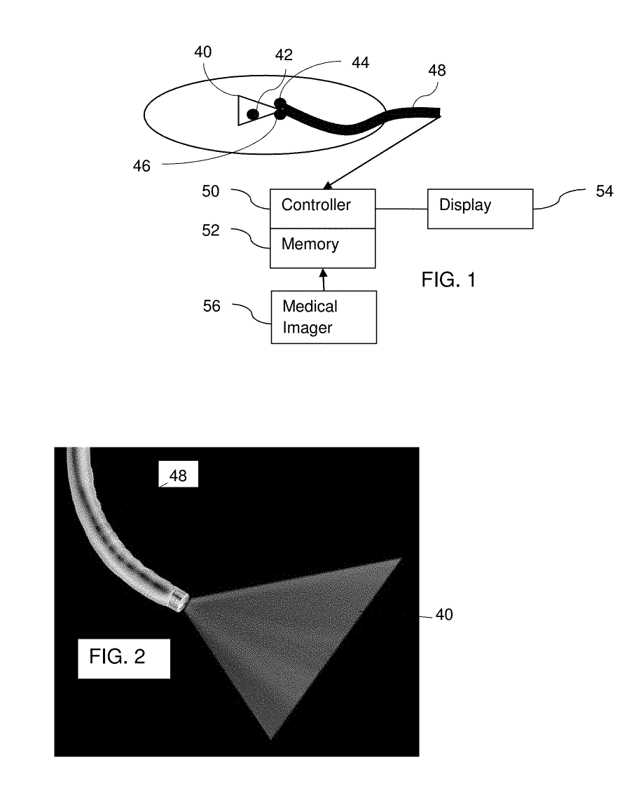

FIG. 1 shows one embodiment of an endoscope system. The endoscope system projects light at tissue where the projected light varies as a function of space and/or time. The variation is controlled to highlight a region of interest, spotlight, set a contrast level, white balance, indicate a path, or provide other information as a projection directly on the tissue. The displayed image has the information from the projection, and the projection is viewable by other imaging devices in the region.

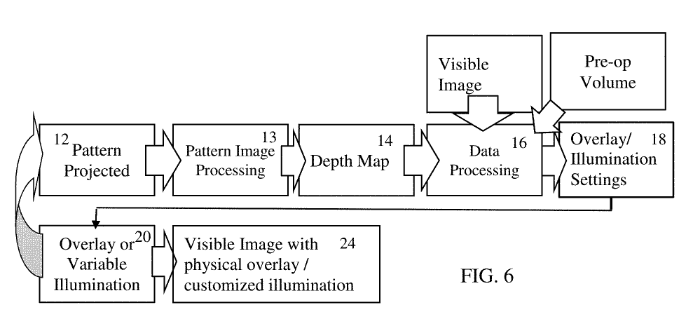

The system implements the method of FIG. 5. Alternatively or additionally, the system implements the method of FIG. 6. Other methods or acts may be implemented, such as projecting light in a spatially varying pattern and capturing an image of the tissue while subjected to the projection, but without the registration or depth mapping operations.

The system includes an endoscope 48 with a projector 44 and a camera 46, a controller 50, a memory 52, a display 54, and a medical imager 56. Additional, different, or fewer components may be provided. For example, the medical imager 56 and/or memory 52 are not provided. In another example, the projector 44 and camera 46 are on separate endoscopes 48. In yet another example, a network or network connection is provided, such as for networking with a medical imaging network or data archival system. A user interface may be provided for interacting with the controller 50 or other components.

The controller 50, memory 52, and/or display 54 are part of the medical imager 56. Alternatively, the controller 50, memory 52, and/or display 54 are part of an endoscope arrangement. The controller 50 and/or memory 52 may be within the endoscope 48, connected directly via a cable or wirelessly to the endoscope 48, or may be a separate computer or workstation. In other embodiments, the controller 50, memory 52, and display 54 are a personal computer, such as desktop or laptop, a workstation, a server, a network, or combinations thereof.

The medical imager 56 is a medical diagnostic imaging system. Ultrasound, CT, x-ray, fluoroscopy, positron emission tomography (PET), single photon emission computed tomography (SPECT), and/or MR systems may be used. The medical imager 56 may include a transmitter and includes a detector for scanning or receiving data representative of the interior of the patient. The medical imager 56 acquires preoperative data representing the patient. The preoperative data may represent an area or volume of the patient. For example, preoperative data is acquired and used for surgical planning, such as identifying a lesion or treatment location, an endoscope travel path, or other surgical information.

In alternative embodiments, the medical imager 56 is not provided, but a previously acquired data set for a patient and/or model or atlas information for patients in general is stored in the memory 52. In yet other alternatives, the endoscope 48 is used to acquire data representing the patient from previous times, such as another surgery or earlier in a same surgery. In other embodiments, preoperative or earlier images of the patient are not used.

The endoscope 48 includes a slender, tubular housing for insertion within a patient. The endoscope 48 may be a laparoscope or catheter. The endoscope 48 may include one or more channels for tools, such as scalpels, scissors, or ablation electrodes. The tools may be built into or be part of the endoscope 48. In other embodiments, the endoscope 48 does not include a tool or tool channel.

The endoscope 48 includes a projector 44 and a camera 46. The projector 44 illuminates tissue of which the camera 46 captures an image while illuminated. An array of projectors 44 and/or cameras 46 may be provided. The projector 44 and camera 46 are at a distal end of the endoscope 48, such as being in a disc-shaped endcap of the endoscope 48. Other locations spaced from the extreme end may be used, such as at the distal end within two to three inches from the tip. The projector 44 and camera 46 are covered by a housing of the endoscope 48. Windows, lenses, or openings are included for allowing projection and image capture.

The projector 44 is positioned adjacent to the camera 46, such as against the camera 46, but may be at other known relative positions. In other embodiments, the projector 44 is part of the camera 46. For example, the camera 46 is a time-of-flight camera, such as a LIDAR device using a steered laser or structured light. The projector 44 is positioned within the patient during minimally invasive surgery. Alternatively, the projector 44 is positioned outside the patient with fiber-optic cables transmitting projections to the tissue in the patient. The cable terminus is at the distal end of the endoscope 28.

The projector 44 is a pico-projector. The pico-projector is a digital light processing device, beam-steering device, or liquid crystal on silicone device. In one embodiment, the projector 44 is a light source with a liquid crystal diode screen configured to control intensity level and/or color as a function of spatial location. In another embodiment, the projector 44 is a steerable laser. Other structured light sources may be used.

The projector 44 is configured by control of the controller 50 to illuminate tissue when the endoscope 48 is inserted within a patient. The tissue may be illuminated with light not visible to a human, such as projecting light in a structured pattern for depth mapping. The tissue may be illuminated with light visible to a human, such as projecting spatially varying light as an overlay on the tissue to be viewed in optical images captured by the camera 46 or otherwise viewed by other viewers. The projected pattern is viewable physically on the tissue.

FIG. 2 illustrates an example projection from the endoscope 48. By using the projector 44 for depth mapping, a fixed or pre-determined pattern is projected during alternating frames captured and not shown to the user. The same or different projector 44 projects overlays and customized lighting during frames shown to the user. The projected light not used for depth mapping may be used for other purposes, such as exciting light-activated drugs and/or to induce fluorescence in certain chemicals. As shown in FIG. 2, the projected light 40 has an intensity and/or color that vary as a function of location output by the projector 44.

The intraoperative camera 46 is a video camera, such as a charge-coupled device (CCD). The camera 46 captures images from within a patient. The camera 46 is on the endoscope 48 for insertion of the camera 46 within the patient's body. In alternative embodiments, the camera 46 is positioned outside the patient and a lens and optical guide are within the patient for transmitting to the camera 46. The optical guide (e.g., fiber-optic cable) terminates at the end of the endoscope 48 for capturing images.

The camera 46 images within a field of view, such as the field of projection 40. A possible region of interest 42 may or may not be within the field of view. The camera 46 is configured to capture an image, such as in a video. The camera 46 is controlled to sense light from the patient tissue. As the tissue is illuminated by the projector 44, such as an overlay or spatial distribution of light in a pattern, the camera 46 captures an image of the tissue and pattern. Timing or other trigger may be used to cause the capture during the illumination. Alternatively, the camera 46 captures the tissue whether or not illuminated. By illuminating, the camera 46 ends up capturing at least one image of the tissue while illuminated.

The memory 52 is a graphics processing memory, a video random access memory, a random access memory, system memory, cache memory, hard drive, optical media, magnetic media, flash drive, buffer, database, combinations thereof, or other now known or later developed memory device for storing data representing the patient, depth maps, preoperative data, image captures from the camera 46, and/or other information. The memory 52 is part of the medical imager 56, part of a computer associated with the controller 50, part of a database, part of another system, a picture archival memory, or a standalone device.

The memory 52 stores preoperative data. For example, data from the medical imager 56 is stored. The data is in a scan format or reconstructed to a volume or three-dimensional grid format. After any feature detection, segmentation, and/or image processing, the memory 52 stores the data with voxels or locations labeled as belonging to one or more features. Some of the data is labeled as representing specific parts of the anatomy, a lesion, or other object of interest. A path or surgical plan may be stored. Any information to assist in surgery may be stored, such as information to be included in a projection (e.g., patient information—temperature or heart rate). Images captured by the camera 46 are stored.

The memory 52 may store information used in registration. For example, video, depth measurements, an image from the video camera 46 and/or spatial relationship information are stored. The controller 50 may use the memory 52 to temporarily store information during performance of the method of FIG. 1 or 2.

The memory 52 or other memory is alternatively or additionally a non-transitory computer readable storage medium storing data representing instructions executable by the programmed controller 50 for controlling projection. The instructions for implementing the processes, methods, and/or techniques discussed herein are provided on non-transitory computer-readable storage media or memories, such as a cache, buffer, RAM, removable media, hard drive, or other computer readable storage media. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The controller 50 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device. The controller 50 is a single device or multiple devices operating in serial, parallel, or separately. The controller 50 may be a main processor of a computer, such as a laptop or desktop computer, or may be a processor for handling some tasks in a larger system, such as in the medical imager 56. The controller 50 is configured by instructions, firmware, design, hardware, and/or software to perform the acts discussed herein.

The controller 50 is configured to control the projector 44 and the camera 46. In one embodiment, the controller 50 controls the projector 44 to project overlaying information for capture by the camera 46 without depth mapping. In another embodiment, the controller 50 also controls the projector 44 to project structured light for depth mapping.

For depth mapping and spatial registration, the controller 50 causes the projector 44 and camera 46 to operate in any now known or later developed registration process. For example, the controller 50 causes the projector 44 to project light in a structured pattern at wavelengths not visible to a human. Visible wavelengths may be used. The structured pattern is a distribution of dots, crossing lines, geometric shapes (e.g., circles or squares), or other pattern.

The specific projected pattern reaches the tissue at different depths. As a result, the pattern intercepted by the tissue is distorted. The controller 50 causes the camera 46 to capture the interaction of the structured light with the tissue. The controller 50 generates a depth map from the captured image of the projected pattern. The controller 50 processes the distortions to determine depth from the camera 46 of tissue at different locations. Any now known or later developed depth mapping may be used.

The controller 50 registers the depth map with a preoperative scan. Using the depth map, the position of the endoscope 48 within the patient, as represented by the preoperative scan, is determined. The depth map indicates points or a point cloud in three-dimensions. The points are correlated with the data of the preoperative scan to find the spatial location and orientation of the depth map with the greatest or sufficient (e.g., correlation coefficient above a threshold) similarity. A transform to align the coordinate systems of the medical imager 56 and the camera 46 is calculated. Iterative closest point, correlation, minimum sum of absolute differences, or other measure of similarity or solution for registration is used to find the translation, rotation, and/or scale that align the data or points in the two coordinate systems. Rigid, non-rigid, or rigid and non-rigid registration may be used.

In one embodiment, additional or different information is used in the registration. For example, an image captured from the camera 46 is used as an independent registration to be averaged with or to confirm registration. The controller 50 compares renderings from the preoperative data or other images with known locations and orientations to one or more images captured by the camera 46. The rendering with the greatest or sufficient similarity is identified, and the corresponding position and orientation information for the rendering provides the location and orientation of the camera 46. Magnetic tracking may be used instead or in addition to other registration. Registration relying on segmentation or landmark identification may be used.

The registration is performed dynamically. Depth maps and/or image capture is repeated. The registration is also repeated. As the endoscope 48 and camera 46 move relative to the patient, the location and orientation derived from the registration is updated. The registration may be performed in real-time during surgery.

The endoscope system alternates projections of a phase-structured light pattern used to compute a depth or distance map image with illumination or data projection used to display the visible image. This alternating prevents the viewer from seeing the structured light used for depth mapping. In other embodiments, the structured light for depth mapping is applied for images that are viewed, but is at non-visible wavelengths. Alternatively, the endoscope system provides for projection for optical viewing without the depth mapping.

The controller 50 is configured to generate an overlay. The overlay is formed as a spatial distribution of light intensity and/or color. For example, one area is illuminated with brighter light than another. As another example, overlaying graphics (e.g., path for movement, region of interest designator, and/or patient information) are generated in light. In yet another example, a rendering from preoperative data is generated as the overlay. Any information may be included in the overlay projected onto the tissue.

In one embodiment, the overlay is generated, in part, from the preoperative scan. Information from the preoperative scan may be used. Alternatively or additionally, the preoperative scan indicates a region of interest. Using the registration, the region of interest relative to the camera 46 is determined and used for generating the overlay.

The projection may be to highlight or downplay anatomy, lesion, structure, bubbles, tool, or other objects. Other objects may be more general, such as projection based on depth. The depth map is used to determine parts of the tissue at different distances from the projector 44 and/or camera 46 and light those parts differently.

To project the highlighting, the controller 50 determines the location of the object of interest. The object may be found by image processing data from the camera 46, from the preoperative scan, from the depth map, combinations thereof, or other sources. For example, computer assisted detection is applied to a captured image and/or the preoperative scan to identify the object. As another example, a template with an annotation of the object of interest is registered with the depth map, indicating the object of interest in the depth map.

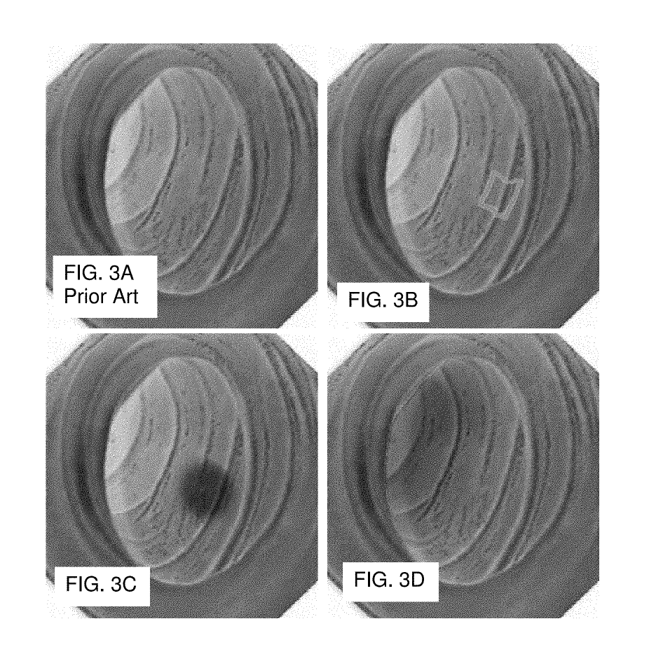

Any relevant data for navigation, guidance, and/or targets may be projected during the visible frame captures. FIG. 3A shows a view of a tubular anatomy structure with a standard endoscope. Uniform illumination or other illumination from a fixed lighting source is applied. Shadows may result. The deeper locations relative to the camera 46 appear darker. A fixed lighting source means that adjustments to the lighting cannot be made without moving the scope or affecting the entire scene. Movements would be necessary to view darker regions, but movements may be undesired.

The controller 50 is configured to control a spatial distribution of illumination from the projector onto tissue. The light is projected by the projector 44 in a pattern. At a given time, the light has different intensity and/or color for different locations. The pattern is an overlay provided on the tissue. Standard endoscopes feature a relatively fixed level of illumination. Regardless of the object being examined and its distance, the illumination is fixed. By allowing spatial control, a wide variety of possibilities for optimal images from the endoscope is provided. Spatial distribution that varies over time and/or location is provided. Rather than a fixed illumination pattern, the projector 44 has a programmable illumination pattern.

In one embodiment, the pattern may be controlled to emphasize one or more regions of interest. A particular region of the image may be spotlighted. FIG. 3C shows an example. Brighter light is transmitted to the region of interest, resulting in a brighter spot as shown in FIG. 3C. Other locations may or may not still be illuminated. For example, the illumination is only provided at the region of interest. As another example, the region is illuminated more brightly, but illumination is projected to other locations. Any relative difference in brightness and/or coloring may be used.

In another embodiment, the illumination projected from the projector 44 is controlled to add more or less brightness for darker regions, such as regions associated with shadow and/or further from the camera 46. For example, the brightness for deeper locations is increased relative to the brightness for shallower locations. FIG. 3D shows an example. This may remove some shadows and/or depth distortion of brightness. In the example of FIG. 3D, the deeper locations are illuminated to be brighter than or have a similar visible brightness as shallower locations. Determining where to move the endoscope 48 may be easier with greater lighting for the deep or more distant locations. Other relative balances may be used, such as varying brightness by depth to provide uniform brightness in appearance.

The color may be controlled based on depth. Color variation across the spatial distribution based on depth may assist a physician in perceiving the tissue. Distances from the camera 46 are color-coded based on thresholds or gradients. Different color illumination is used for locations at different depths so that the operator has an idea how close the endoscope 48 is to structures in the image. Alternatively or additionally, surfaces more or less orthogonal to the camera view are colored differently, highlighting relative positioning. Any color map may be used.

In one embodiment, the controller 50 causes the projector 44 to illuminate the region of interest with an outline. Rather than or in addition to the spot lighting (see FIG. 3C), an outline is projected. FIG. 3B shows an example. The outline is around the region of interest. The outline is formed as a brighter line or a line projected in a color (e.g., green or blue). By illuminating in a different color, the region may be highlighted. Based on determining the location of the region, the region is highlighted with a spotlight, border, or other symbol. The spotlight may be colored, such as shaded in green. Other highlighting or pointing to the region of interest may be used, such as projecting a symbol, pointer, or annotation by the region.

Since a projector provides lighting, any type of illumination control or graphics are possible. Other graphics, such as text, measurements, or symbols, may be projected based on or not based on the region of interest. Unlike a conventional post-processing blended overlay, the graphics are actually projected onto the tissue and visible to any other devices in the region.

In one embodiment, the spatial distribution of illumination is controlled to reduce intensity at surfaces with greater reflectance than adjacent surfaces. The color, shade and/or brightness may be used to reduce glare or other undesired effects of capturing an image from reflective surfaces. The coloring or brightness for a reflective surface is different than used for adjacent surfaces with less reflectance. Eliminating excessive reflection due to highly reflective surfaces, such as bubbles, may result in images from the camera 46 that are more useful. For example, “bubble frames” may be encountered during airway endoscopy. In such frames, a bubble developed from the patient's airways produces reflections in the acquired image to the point of making that particular image useless for the operator or any automated image-processing algorithm. Bubbles are detected by image processing. The locations of the detected bubbles are used to control the projection. By lighting the bubbles with less intense light or light shaded by color, the resulting images may be more useful to computer vision algorithms and/or the operator.

The pattern of light from the projector may vary over time. As the region of interest relative to the camera 46 or projector 44 shifts due to endoscope 48 or patient motion, the controller 50 determines the new location. The projector 44 is controlled to alter the pattern so that the illumination highlighting the region shifts with the region. The registration is updated and used to determine the new location of the region of interest. Other time varying patterns may be used, such as switching between different types of overlays being projected (e.g., every second switching from highlighting one region to highlighting another region). Text, such as patient measure, may change over time, so the corresponding projection of that text changes. Due to progression of the endoscope 48, a graphic of the path may be updated. Due to movement of the endoscope 48, a different image rendered from the preoperative data may result and be projected onto the tissue.

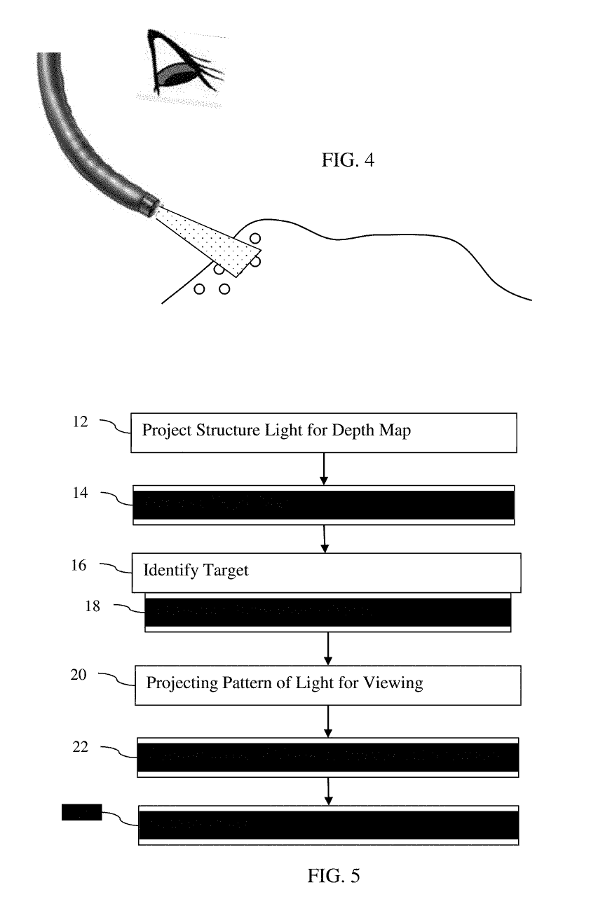

In another embodiment, the controllable illumination is used for drug activation or release. The spatial distribution and/or wavelength (i.e., frequency) is altered or set to illuminate one or more regions of interest where drugs are to be activated. Light activated drugs activate the release or cause a chemical reaction when exposed to light of certain frequencies. Light at frequencies to which the drug activation is insensitive may be used to aid guidance of the endoscope or for any of the overlays while light to which the drug activation is sensitive may be projected in regions where drug release is desired. FIG. 4 shows an example where the circles represent drug deposited in tissue. The beam or illumination at drug activation frequencies is directed to the tissue location where treatment is desired and not other locations. The use of the real-time registration allows the endoscope to adjust for any jitter movements from the operator and/or patient to avoid drug release or activation where not desired.

In addition, due to the flexibility of frequency and spatial distribution of illumination of the projector 44, the operator may guide the endoscope to the region and release control of the device to stabilize the illumination. The registration is regularly updated so that the region for activation is tracked despite tissue movement or other movement of the endoscope 48 relative to the tissue. The controller 50 controls the projector 44 to target the desired location without further user aiming. The user may input or designate the region of interest in an image from the camera 46 and/or relative to the preoperative volume.

Other applications or uses of the controllable lighting may be used. Where visible wavelengths are used to generate a visible overlay, other cameras on other devices or other viewers in an open surgery (e.g., surgery exposing the tissue to the air or direct viewing from external to the patient) may perceive the overlay. The projection provides an overlay visible to all other viewers. FIG. 4 shows an example where a viewer watches the illuminated tissue during drug activation, so may monitor that the drugs are activated at the desired location. The secondary viewer may directly view any overlay on the tissue or objects in the physical domain rather than just a processed display. This capability is impossible to achieve using an artificial overlay of an image rather than light projected on the tissue.

Using user-interface control of the overlay, the endoscopist may point to the video feed (e.g., select a location on an image) and have that point or region highlighted in reality on the tissue to the benefit of other tools or operators. Similarly, computer assisted detection may identify a region and have that region highlighted for use by other devices and/or viewers.

In alternative or additional embodiments, the controller 50 is configured to control the spatial distribution for contrast compensation of the camera 46. The sensitivity of the light sensor forming the camera 46 may be adjusted for the scene to control contrast. To assist or replace this operation, the illumination may be controlled so that the sensitivity setting is acceptable. The lighting of the scene itself is adjusted or set to provide the contrast. The CCD or other camera 46 and the lighting level may both be adjusted. The light level and regions of illumination are set, at least in part, to achieve optimal image contrast.

Similarly, the controller 50 may control the spatial distribution of color in the projection for white balance in the image. The illumination is set to provide white balance to assist in and/or to replace white balancing by the camera 46.

It is common in CCD imaging systems to automatically adjust contrast and white balance on the sensor depending upon the scene being imaged. This allows for a more consistent image and color renditions across different scenes and light sources. In standard endoscopes, it may be preferred to manually adjust white balance based on a calibration image such as a white sheet of paper. The brightness or projection characteristics may be another variable in the process of automated white balancing. For example, both the color and intensity of the light in the environment are adjusted to provide some or all of the white balance and/or contrast.

In other embodiments, combinations of different applications or types of overlays are projected. For example, the controller 50 controls the projector 44 to highlight one or more regions of interest with color, graphics, and/or shading while also illuminating the remaining field of view with intensity variation for contrast and/or white balance. Once triggered, other illumination at a different frequency is applied to activate drugs. In another example, brighter light is applied to deeper regions while light directed at surfaces with greater reflectivity is reduced to equalize brightness over depth and reflectivity of surfaces. Any combination of overlays, light pattern, and/or spatial variation of intensity or color may be used.

In other embodiments, the focus of the projector 44 may be automatically adjusted based on the core region of interest using the depth information. Alternatively, focus-free projection technology such as those offered by LCOS panels in pico projectors is used.

Referring again to FIG. 1, the display 54 is a monitor, LCD, projector, plasma display, CRT, printer, or other now known or later developed device for displaying the image from the camera 46. The display 54 receives images from the controller 50, memory 52, or medical imager 56. The images of the tissue captured by the camera 46 while illuminated with the overlay pattern by the projector 44 are displayed. Other information may be displayed as well, such as controller generated graphics, text, or quantities as a virtual overlay not applied by the projector 44.

Additional images may be displayed, such as a rendering from a preoperative volume to represent the patient and a planned path. The images are displayed in sequence and/or side-by-side. The images use the registration so that images representing a same or similar view are provided from different sources (e.g., the camera 46 and a rendering from the preoperative volume).

FIG. 5 shows a flow chart of one embodiment of a method for projection in medical imaging. Light viewable in a captured image is applied to tissue. The light is patterned or structured to provide information useful for the surgery. The pattern is an overlay.

FIG. 6 shows another embodiment of the method. FIG. 6 adds the pattern processing used to create the depth map as well as indicating sources of data for determining the illumination pattern.

The methods are implemented by the system of FIG. 1 or another system. For example, some acts of one of the methods are implemented on a computer or processor associated with or part of an endoscopy, computed tomography (CT), magnetic resonance (MR), positron emission tomography (PET), ultrasound, single photon emission computed tomography (SPECT), x-ray, angiography, or fluoroscopy imaging system. As another example, the method is implemented on a picture archiving and communications system (PACS) workstation or implemented by a server. Other acts use interaction with other devices, such as the camera and/or projector. The projector performs acts 12 and 20.

The acts are performed in the order shown or other orders. For example, acts 16-24 may be performed before acts 12-14. In an alternating or repeating flow switching between acts 12-14 and 16-22, act 24 may be performed before, after, or simultaneously with any of the other acts.

In the embodiments of FIGS. 5 and 6, the projections are used for both depth mapping to register and applying an overlay. Acts 12-14 are performed for registration while acts 16-24 are performed for physically projecting an overlay. The projector alternates between projecting the pattern of structured light for depth mapping and projecting the pattern of structured light as an overlay. The projection for depth mapping and generating of the depth map and corresponding registration alternates with the identification of the target or target location, projecting of the pattern to the target, capturing the pattern in the image, and displaying the image with the pattern. It is also possible to produce a structured light pattern suitable for computing a depth map yet offering a unique pattern that would be suitable for an overlay. Such a setting allows for increased frame rates as alternating may be avoided, allowing the endoscope to be used for high-speed applications.

Additional, different, or fewer acts may be provided. For example, acts 12-14 are not performed. The images from the camera on the endoscope are used to identify a target for spatially controlled illumination. As another example, act 16 is not provided where the illumination pattern is based on other information, such as projecting a rendered image, projecting patient information not specific to a region or target, or projecting a pattern based on the depth map.

In one embodiment, a preoperative volume or scan data may be used to assist in surgery. A region or regions of interest may be designated in the preoperative volume as part of planning. A path may be designated in the preoperative volume as part of planning. Renderings from the preoperative volume may provide information not available through images captured by the camera on the endoscope.

Any type of scan data may be used. A medical scanner, such as a CT, x-ray, MR, ultrasound, PET, SPECT, fluoroscopy, angiography, or other scanner provides scan data representing a patient. The scan data is output by the medical scanner for processing and/or loaded from a memory storing a previously acquired scan.

The scan data is preoperative data. For example, the scan data is acquired by scanning the patient before the beginning of a surgery, such as a minutes, hours, or days before. Alternatively, the scan data is from an intraoperative scan, such as scanning while minimally invasive surgery is occurring.

The scan data, or medical imaging data, is a frame of data representing the patient. The data may be in any format. While the term “image” is used, the image may be in a format prior to actual display of the image. For example, the medical image may be a plurality of scalar values representing different locations in a Cartesian or polar coordinate format the same as or different than a display format. As another example, the medical image may be a plurality red, green, blue (e.g., RGB) values to be output to a display for generating the image in the display format. The medical image may be currently or previously displayed image in the display format or other format.

The scan data represents a volume of the patient. The patient volume includes all or parts of the patient. The volume and corresponding scan data represent a three-dimensional region rather than just a point, line or plane. For example, the scan data is reconstructed on a three-dimensional grid in a Cartesian format (e.g., N×M×R grid where N, M, and R are integers greater than one). Voxels or other representation of the volume may be used. The scan data or scalars represent anatomy or biological activity, so is anatomical and/or functional data.

To determine the position and orientation of the endoscope camera relative to the preoperative volume, sensors may be used, such as ultrasound or magnetic sensors. In one embodiment, acts 12 and 14 are used to register the position and orientation of the camera relative to the preoperative volume.

In act 12, a projector projects a pattern of structured light. Any pattern may be used, such as dots, lines, and/or other shapes. The light for depth mapping is at a frequency not viewable to humans, but may be at a frequency viewable to humans. The pattern is separate from any pattern used for viewing. In alternative embodiments, the overlay is used as the pattern for depth mapping.

The projected light is applied to the tissue. Due to different depths of the tissue relative to the projector, the pattern appears distorted as captured by the camera. This distortion may be used to determine the depth at different pixels or locations viewable by the camera at that time in act 13. In other embodiments, the depth measurements are performed by a separate time-of-flight (e.g., ultrasound), laser, or other sensor positioned on the intraoperative probe with the camera.

In act 14, a depth map is generated. With the camera inserted in the patient, the depth measurements are performed. As intraoperative video images are acquired or as part of acquiring the video sequences, the depth measurements are acquired. The depths of various points (e.g., pixels or multiple pixel regions) from the camera are measured, resulting in 2D visual information and 2.5D depth information. A point cloud for a given image capture is measured. By repeating the capture as the patient and/or camera move, a stream of depth measures is provided. The 2.5D stream provides geometric information about the object surface and/or other objects.

A three-dimensional distribution of the depth measurements is created. The relative locations of the points defined by the depth measurements are determined. Over time, a model of the interior of the patient is created from the depth measurements. In one embodiment, the video stream or images and corresponding depth measures for the images are used to create a 3D surface model. The processor stiches the measurements from motion or simultaneous localization and mapping. Alternatively, the depth map for a given time based on measures at that time is used without accumulating a 3D model from the depth map.

The model or depth data from the camera may represent the tissue captured in the preoperative scan, but is not labeled. To align the coordinate systems of the preoperative volume and camera, a processor registers the coordinate systems using the depth map and/or images from the camera and the preoperative scan data. For example, the three-dimensional distribution (i.e., depth map) from the camera is registered with the preoperative volume. The 3D point cloud reconstructed from the intraoperative video data is registered to the preoperative image volume. As another example, images from the camera are registered with renderings from the preoperative volume where the renderings are from different possible camera perspectives.

Any registration may be used, such as a rigid or non-rigid registration. In one embodiment, a rigid, surface-based registration is used. The rotation, translation, and/or scale that results in the greatest similarity between the compared data is found. Different rotations, translations, and/or scales of one data set relative to the other data set are tested and the amount of similarity for each variation is determined. Any measure of similarity may be used. For example, an amount of correlation is calculated. As another example, a minimum sum of absolute differences is calculated.

One approach for surface-based rigid registration is the common iterative closest point (ICP) registration. Any variant of ICP may be used. The depth map represents a surface. The surfaces of the preoperative volume may be segmented or identified.

Once registered, the spatial relationship of the camera of the endoscope relative to the preoperative scan volume is known. Acts 12-14 may be repeated regularly to provide real-time registration, such as repeating every other capture by the camera or 10 Hz or more.

In act 16, the processor identifies one or more targets in a field of view of the endoscope camera. The target may be the entire field of view, such as where a rendering is to be projected as an overlay for the entire field of view. The target may be only a part of the field of view. Any target may be identified, such as a lesion, anatomy, bubble, tool, tissue at deeper or shallower depths, or other locations. The targets may be a point, line, curve, surface, area, or other shape.

The user identifies the target using input, such as clicking on an image. Alternatively or additionally, computer-assisted detection identifies the target, such as identifying suspicious polyps or lesions. An atlas may be used to identify the target.

The target is identified in an image from the camera of the endoscope. Alternatively or additionally, the target is identified in the preoperative scan.

In act 18, a processor determines an illumination pattern. The pattern uses settings, such as pre-determined or default selection of the technique (e.g., border color, spotlight, shading, or combinations thereof) to highlight a region of interest. Other settings may include the contrast level. The pattern may be created based on input information from the user and the settings. The pattern may be created using feedback measures from the camera. Alternatively or additionally, the pattern is created by selecting from a database of options. The pattern may be a combination of different patterns, such as providing highlighting of one or more regions of interest as well as overlaying patient information (e.g., heart rate).

The depth map may be used. Different light intensity and/or color are set as a function of depth. The contrast and/or white balance may be controlled, at least in part, through illumination. The depth is used to provide variation for more uniform contrast and/or white balance. Segmentation of the preoperative volume may be used, such as different light for different types of tissue visible by the endoscope camera.

In one embodiment, the depth map is used to distort the pattern. The distortion caused by depth (e.g., the distortion used to create the depth map) may undesirably distort the highlighting. The pattern is adjusted to counteract the distortion. Alternatively, the distortion is acceptable, such as where the target is spotlighted.

In one embodiment, the registration is used to determine the pattern. The target is identified as a region of interest in the preoperative volume. The registration is used to transform the location in the preoperative volume to the location in the camera space. The pattern is set to illuminate the region as that target exists in the tissue visible to the camera.

In act 20, the projector projects the pattern of light from the endoscope. The pattern varies in color and/or intensity as a function of location. The tissue is illuminated with the pattern. The target, such as a region of interest, is illuminated differently than surrounding tissue in the field of view. The illumination highlights the target. For example, a bright spot is created at the target. As another example, a colored region and/or outline is created at the target. In yet another example, contrast or white balance is created at the target (e.g., deeper depths relative to the camera). As another example, the pattern includes light to activate drug release or chemical reaction at desired locations and not at other locations in the field of view.

In act 22, the camera captures one or more images of the tissue as illuminated by the pattern. The endoscope generates an image of the field of view while illuminated by the projection. When the illumination is in the visible spectrum, the overlay is visible in both the captured image as well as to other viewers. Any overlay information may be provided.

In act 24, the captured image is displayed on a display. The image is displayed on a display of a medical scanner. Alternatively, the image is displayed on a workstation, computer, or other device. The image may be stored in and recalled from a PACS or other memory.

The displayed image shows the overlay provided by the projected illumination. Other images may be displayed, such as a rendering from the preoperative volume displayed adjacent to but not over the image captured by the camera. In one embodiment, a visual trajectory of the medical instrument is provided in a rendering of the preoperative volume. Using the registration, the pose of the tip of the endoscope is projected into a common coordinate system and may thus be used to generate a visual trajectory together with preoperative data. A graphic of the trajectory as the trajectory would be seen if a physical object is projected so that the image from the endoscope shows a line or other graphic as the trajectory.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. An endoscope system comprising:
a projector optically connected with an endoscope;
a controller configured to control a spatial distribution of illumination from the projector onto tissue in a first pattern;
a camera on the endoscope, the camera configured to capture an image of patient tissue as illuminated by the spatial distribution of the first pattern; and
a display configured to display the image from the camera.

2. The endoscope system of claim 1 wherein the projector and the camera are operable to image and project from a distal end of the endoscope.

3. The endoscope system of claim 1 wherein the projector comprises a pico-projector.

4. The endoscope system of claim 1 wherein the camera comprises a charge-coupled device.

5. The endoscope system of claim 1 wherein the controller is configured to image process data from the camera, from a preoperative scan, a depth map or combinations thereof, the image process identifying a region of interest in a field of view of the camera and is configured to control the spatial pattern to emphasize the region of interest.

6. The endoscope system of claim 1 wherein the controller is configured to control the spatial distribution for contrast compensation of the camera.

7. The endoscope system of claim 1 wherein the controller is configured to control the spatial distribution for white balance in the image.

8. The endoscope system of claim 1 wherein the controller is configured to control the spatial distribution to apply the illumination at a drug-activation frequency to a treatment location.

9. The endoscope system of claim 1 wherein the controller is configured to control the spatial distribution such that the first pattern is an outline of a region of interest.

10. The endoscope system of claim 1 wherein the controller is configured to control the spatial distribution such that the first pattern has intensity variation as a function of distance of tissue from the projector.

11. The endoscope system of claim 1 wherein the controller is configured to control color variation across the spatial distribution as a function of depth to the patient tissue.

12. The endoscope system of claim 1 wherein the controller is configured to control the spatial distribution of the illumination to reduce intensity at surfaces with greater reflectance than adjacent surfaces, the reduction relative to the illumination at the adjacent surfaces with the lesser reflectance.

13. The endoscope system of claim 1 wherein the controller is configured to control the spatial distribution of illumination into a second pattern, the second pattern using wavelengths not visible to a human, is configured to generate a depth map from a capture from the camera of the interaction of the second pattern with the tissue, is configured to register the depth map with a preoperative scan, and is configured to generate an overlay from the preoperative scan based on the registration, the overlay being on the image.

14. The endoscope system of claim 13 wherein the controller is configured to adjust the first pattern to illuminate a region of interest despite movement using the registration.

15. The endoscope system of claim 1 wherein the illumination from the first pattern is viewable physically on the tissue.

16. The endoscope system of claim 1 wherein the first pattern changes over time.

17. A method for projection in medical imaging, the method comprising:
identifying a target in a field of view of an endoscope;
illuminating, by the endoscope, the target differently than surrounding tissue in the field of view; and
generating an image by the endoscope of the field of view while the target is illuminated.

18. A method for projection in medical imaging, the method comprising:
projecting a first pattern of structured light from an endoscopic device;
generating by the endoscopic device a depth map using captured data representing the first pattern of structured light;
projecting a second pattern of light from the endoscopic device, the second pattern varying color, intensity, or color and intensity as a function of location;
capturing an image of tissue as illuminated by the second pattern; and
displaying the image;
wherein the projecting of the first pattern and generating alternate with the projecting of the second pattern, capturing, and displaying.

19. The method of claim 18 further comprising determining the second pattern from preoperative data spatially registered with the endoscopic device using the depth map.

20. The method of claim 18 further comprising determining the second pattern as a function of level of contrast.
